(19) United States
(12) Patent Application Publication
(10) Pub. No.: US 2024/0366380 A1
(43) Pub. Date: Nov. 7, 2024

(54) ARTICULATED PROSTHESIS FOR A TRICUSPID OR MITRAL VALVE AND RELATED CATCHING DEVICE

(71) Applicant: STAR TRIC S.R.L., Milano (IT)

(72) Inventors: Marco GARD, Borgomasino (TO) (IT); Matteo ANTONIOTTI, Settimo Torinese (TO) (IT); Daniele ZANOTTI, Comelico Superiore (BL) (IT); Michele DE BONIS, Cernusco Sul Naviglio (MI) (IT); Eugenio Maria PASSANANTE, Milano (MI) (IT)

(73) Assignee: STAR TRIC S.R.L., Milano (IT)

(21) Appl. No.: 18/560,975

(22) PCT Filed: May 17, 2022
(86) PCT No.: PCT/IB2022/054572

§ 371 Date: Nov. 15, 2023

(30) Foreign Application Priority Data
May 17, 2021 (IT) 102021000012578

(51) Int. Cl. A61F 2/24

(52) U.S. Cl. CPC A61F 2/246; A61F 2/2466; A61F 2220/0008

(57) ABSTRACT

A device for joining together the flaps of a tricuspid valve by joining them together while they arm kept aligned along the valve plane, is provided with a plurality of arms each having a first end fixed on a cylindrical proximal body and a second end fixed to a cylindrical distal body, in which each arm is made of shape memory material and is configured to assume a radially expanded position with an elbow bend so that, when they are in said radially expanded position with an elbow bend, they define as many radially oriented coplanar supports to support flaps of a tricuspid or mitral valve.

### TECHNICAL FIELD

This disclosure relates to the repair of heart valves showing regurgitation. More particularly, the invention relates to an apparatus suitable for a less invasive repair of a heart valve using an articulated prosthesis of a catching device, that may be positioned through a catheter, for catching the flaps of a tricuspid or mitral valve.

### BACKGROUND

The most common type of tricuspid valve dysfunction is functional tricuspid regurgitation (TR), which is mainly due to dilation of the tricuspid annulus after the dilation of the right ventricle. Later in the course of the disease, the “tethering” of the tricuspid flaps may also take place due to the dislocation of the papillary muscles within the remodeled right ventricle. When the functional TR is due to both severe annular dilation and flap chaining, annuloplasty alone is unlikely to be effective. Similarly. TR caused by prolapse or “flail” of multiple flaps, as typically seen in post-traumatic TR and severe degenerative TR, cannot be corrected by a simple annuloplasty procedure.

To obtain an effective and lasting repair, the so-called “clover” technique has been proposed. This technique consists of tying together the central part of the free edges of the tricuspid flaps, producing a valve in the shape of a “clover”. A visual representation of a tricuspid valve treated according to this technique is shown in FIG. 1. Clinical results obtained with this technique are reported in:
“The ‘clover technique’ as a novel approach for correction of post-traumatic tricuspid regurgitation”, O. Alfieri, M. De Bonis et al., Journal of Thoracic and Cardiovascular Surgery, 2003; Vol. 126, No. 1, pages 75-79;
“A novel technique for correction of severe tricuspid valve regurgitation due to complex lesions.” De Bonis M, Lapenna E et al. Eur. J. Cardiothorac. Surg. 2004 May; 25(5):760-5.
“Four-leaflet clover repair of severe tricuspid valve regurgitation due to complex lesions”, E. Lapenna, M. De Bonis et al., Journal of Cardiovascular Medicine, 2008, Vo. 9 No. 8, pages 847-849;
“The clover technique for the treatment of complex tricuspid valve insufficiency: midterm clinical and echocardiographic results in 66 patients”, E. Lapenna, M. De Bonis et al., European Journal of Cardio-thoracic Surgery, 37 (2010), 1297-1303;
“Long-term results (up to 14 years) of the clover technique for the treatment of complex tricuspid valve regurgitation”, De Bonis M, Lapenna E, et al. Eur. J. Cardiothorac. Surg. 2017 Feb. 23. doi: 10.1093/ejcts/ezx027.

Devices for catching opposite flaps of a mitral valve as well as a tricuspid valve are sold under the trade names MITRACLIP™ and TRICLIP™. This known device, which can be introduced into the heart through a catheter with a vascular approach or through a small incision in the chest, comprises an applicator of a catching device of the type shown in FIG. 2. The sequence of operations to be performed to implant a device MITRACLIP™ catching device is shown in FIG. 3. In the illustrated case the heart valve is the mitral valve, but the same observations apply mutatis mutandis also to the tricuspid valve. Using a catheter, the MITRACLIP™ catching device is inserted in a bent configuration into the heart; when the catheter is close to the heart valve, the latch is deployed like an umbrella to capture the valve flaps, and is subsequently closed to hold the flaps together. Finally, the MITRACLIP™ catching device is left closed in the heart to hold the flaps together, thereby reducing valve regurgitation.

Unfortunately, tests performed by the Applicant have shown that this known applicator is not capable of simultaneously catching all three flaps of the tricuspid valve, therefore, its effectiveness in treating tricuspid regurgitation is very limited. In the presence of a highly dilated tricuspid annulus, catching only two tricuspid valve flaps is also difficult with the MITRACLIP™ system.

On the other hand, it would be desirable to have a device that allows the flaps of the tricuspid (or mitral) valve to be connected to each other exactly as shown in FIG. 9, keeping the flaps coplanar.

### SUMMARY

An objective of this disclosure is to provide a device for joining the flaps of a tricuspid valve together by tying them together while being held aligned along the valve plane.

This outstanding result is achieved with a device as defined in the appended claim 1, which is provided with a plurality of arms each having a first end fixed on a cylindrical proximal body and a second end fixed to a cylindrical distal body, in which each arm is made of shape memory material and is configured to assume a radially expanded position with an elbow bend so that, when they are in said radially expanded position with an elbow bend, they define as many coplanar supports radially oriented to sustain the flaps of a heart valve, e.g. tricuspid or mitral valve.

Further embodiments are defined in the attached claims.

### BRIEF DESCRIPTION OF THE DRAWINGS

FIG. 1 schematically shows a typical configuration of a tricuspid valve after surgery with the so-called “clover” technique.

FIG. 2 shows a known attachment device for flaps of a tricuspid or mitral valve.

FIG. 3 shows various steps for implanting the so-called MITRACLIP™ catching device to the flaps of a heart valve.

FIGS. 4a and 4b are views taken from different points of a device of this disclosure in closed configuration, with unbent arms.

FIGS. 5a and 5b are views taken from different points of a device of this disclosure in an unbent configuration, with the arms bent to form an elbow to define a resting plane for heart valve flaps.

FIGS. 6a and 6b are views taken from different points of a device of this disclosure in an unbent configuration and with suture threads emerging from lateral holes of the cylindrical proximal body of the device.

FIG. 6c is a sectional view of the device of FIGS. 6a and 6b showing a suture thread that emerges from a through channel in the cylindrical proximal body.

FIGS. 7a and 7b show the device of FIGS. 4a and 4b inserted between the flaps of a tricuspid valve.

FIGS. 8a and 8b show the device of FIGS. 5a and 5b inserted between the flaps of a tricuspid valve, with the arms bent to form an elbow which define a support plane for the flaps of the tricuspid valve.

FIGS. 9a and 9b show the device of FIGS. 6a and 6b inserted between the flaps of a tricuspid valve, with the suture threads crossing the flaps of the tricuspid valve.

FIGS. 10a and 10b show the suture threads connected to the flaps of a tricuspid valve positioned using the device of FIGS. 9a and 9b.

FIGS. 11a and 11b show a device for cutting and holding a plurality of suture threads according to the present disclosure, usable with the device of FIGS. 6a and 6b.

FIGS. 12a to 12d show how to position the cutting and holding device of FIGS. 11a and 11b to stretch the suture threads in the center of a tricuspid valve.

FIG. 12e is a top view of a countersunk washer of the cutting and holding device of FIGS. 11a to 12d, positioned in the center of a tricuspid valve.

FIGS. 13a to 13g show how to cut and hold the suture threads using the cutting and holding device of FIGS. 11a and 11b.

FIGS. 14a to 14c show how the suture threads are cut and held thanks to the cooperation between a screw and a countersunk washer having a central through hole with a nut screw thread of the cutting and holding device of FIGS. 11a and 11b.

FIGS. 15a to 15d show hinged arms with an elbow joint which may be bent by rotating around the joint to define a resting plane of the flaps of a heart valve, usable in the catching device of FIGS. 5a and 5b.

FIGS. 16a to 16f show hinged arms with an elbow joint and a free protruding end which may be bent by rotating around the joint until they assume a hook shape which may be used in the catching device of FIGS. 5a and 5b so as to tighten the flaps of a heart valve against the cylindrical proximal body 3.

FIGS. 17a to 17f show integral arms that may be elastically deformed until they assume a hook shape that may be used in the catching device of FIGS. 5a and 5b so as to tighten the flaps of a heart valve against the cylindrical proximal body 3.

FIG. 18a shows an anchoring element inside a supporting catheter (in semitransparency) in a longitudinally extended configuration.

FIG. 18b shows an anchoring element on the outside of a supporting catheter in a radially expanded configuration.

FIGS. 19a and 20a show anchoring elements in a longitudinally extended configuration obtained through longitudinal cuts on a side wall of an elastic or shape memory tubular element.

FIGS. 19b and 20b show anchoring elements in a radially expanded configuration.

FIG. 21a is a proximal view of the anchor element of FIG. 19b connected to a suture thread.

FIG. 21b is a sectional view of the anchor element of FIG. 21a showing the suture thread embedded in a solid resin block and this solid resin block is interlocked to the internal walls of the anchor by means of a solid central strut.

FIGS. 22a to 22d show a section of a cutting and holding device for cutting and holding the suture threads, usable with the device of FIG. 6a, based on the cooperation between a screw and a countersunk washer having a central through hole with a thread nut and a device blade that can rotate circumferentially.

FIGS. 23a to 23d show a detail of another cutting and holding device similar to that of FIGS. 22a to 22d, in which the screw stem is also internally threaded, with a threaded pin screwed into the screw and in the washer.

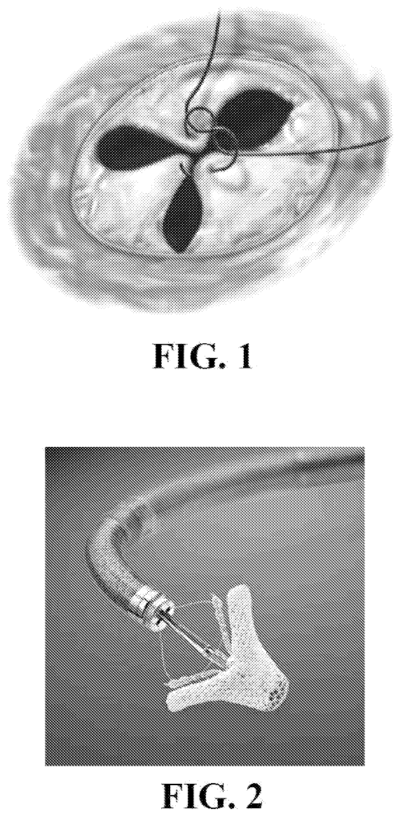

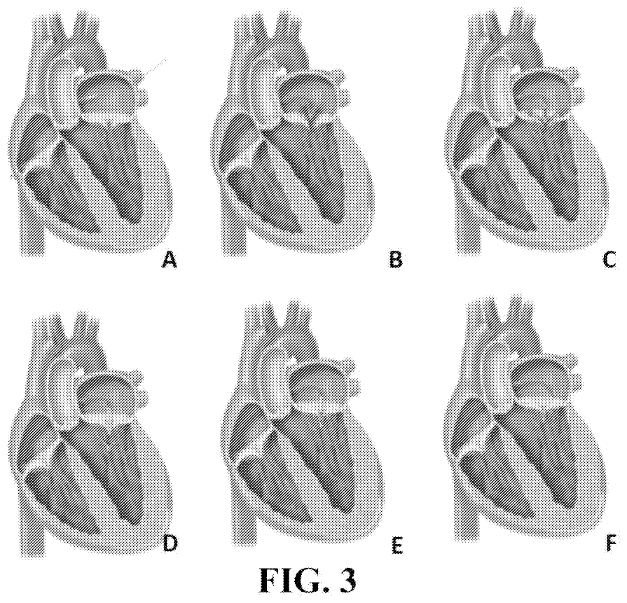

### DESCRIPTION OF EXEMPLARY EMBODIMENTS

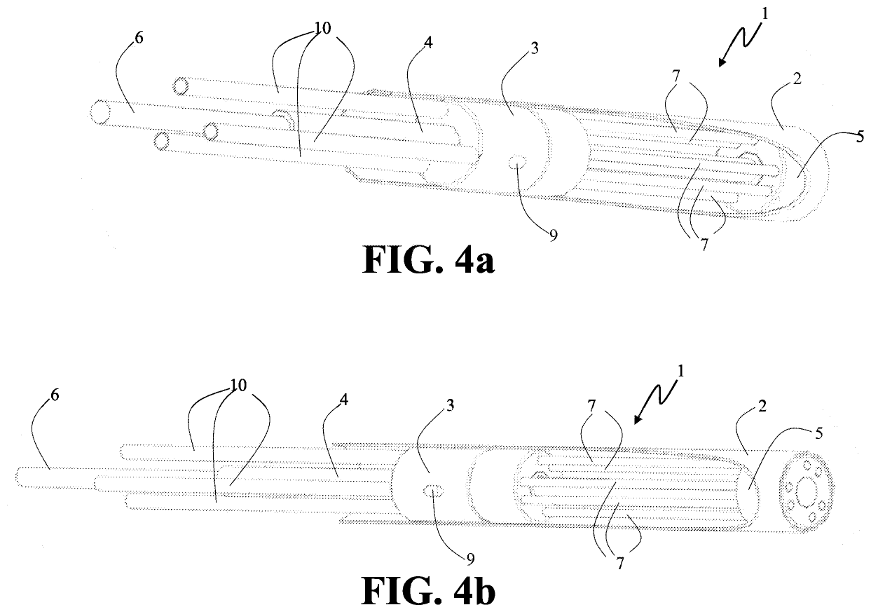

An embodiment of a device for joining together the flaps of a tricuspid valve, or even of a mitral valve, will be illustrated with reference to FIGS. 4a to 6c.

The illustrated device 1 comprises an external catheter 2 suitable for being inserted into a patient's blood vessel, that performs the function of a container which carries inside the blood vessel the internal part for joining the flaps together. The internal part comprises a cylindrical proximal body 3, which defines a central axial cavity 4 which runs in the cylindrical proximal body 3 from a first base surface to a second base surface, as well as a cylindrical distal body 5, which has a rigid coaxial shaft 6 which is fixed and rises from a central position of a base surface of the cylindrical distal body 5 and which is slidably inserted into the central axial cavity 4 of the cylindrical proximal body 3. In practice, by pushing or pulling the rigid coaxial shaft 6 it is possible to move the cylindrical distal body 5 away or closer to the cylindrical proximal body 3.

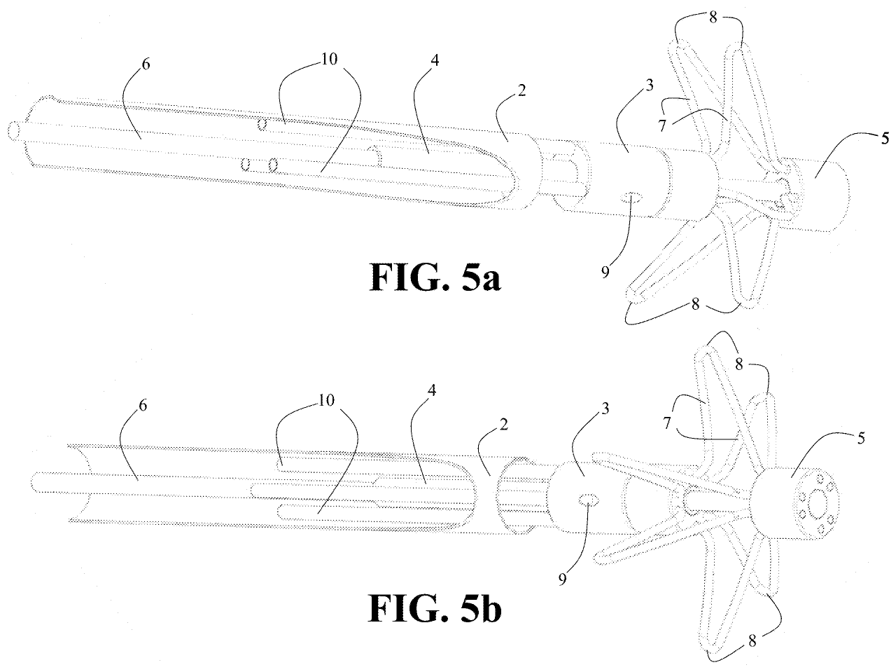

Between the two cylindrical bodies 3 and 5, several flexible arms 7 are installed which have a first end fixed on a base surface of the cylindrical proximal body, and a second end fixed to the cylindrical distal body. Each arm is made of shape memory material and is configured to spontaneously assume a radially expanded position with an elbow 8 when exiting the external catheter 2, as shown in FIGS. 5a and 5b, and to assume an extended position parallel to the rigid coaxial shaft 6 when both the cylindrical proximal body 3 and the cylindrical distal body 5 are inside the external catheter 2, as shown in FIGS. 4a and 4b.

The arms 7 are configured in such a way as to define, when they are in the radially expanded position, coplanar supports radially oriented to support the flaps of a tricuspid or mitral valve, keeping them substantially aligned with the plane identified by the supports.

In the cylindrical proximal body 3 there are at least two through channels 9 which run in the cylindrical proximal body 3 from as many inlet holes in the first base surface of the cylindrical proximal body 3 to as many outlet holes.

In the embodiment illustrated in FIGS. 4a to 6c, the outlet holes of the through channels are located in the lateral surface of the cylindrical proximal body 3.

Each of these through channels 9 is connected to a respective internal catheter 10, so as to be able to convey a suture thread through the internal catheter 10 and to direct it at least partially in the radial direction making it come out of the respective outlet hole from the lateral surface of the cylindrical proximal body 3.

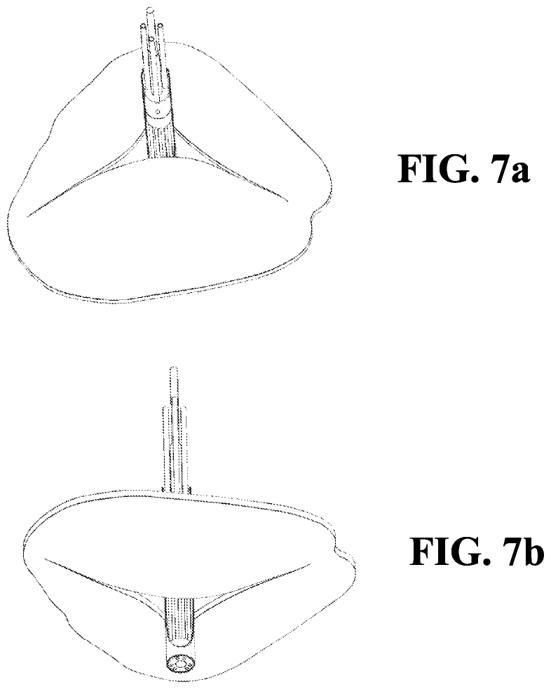

To better understand how the device of FIGS. 4a to 6c of this disclosure is used to join together the flaps of a heart valve, reference will be made to FIGS. 7a to 9c which show the use of the device 1 to join the three flaps of a tricuspid valve.

The closed device 1 is pushed through a blood vessel until it reaches the heart valve whose flaps must be tied together, positioning it so that the external catheter 2 crosses the valve plane and the opening of the external catheter 2 is beyond this plane, so as to open below the flaps to be tied together, as shown in FIGS. 7a and 7b. Keeping the external catheter 2 in this position, the rigid coaxial shaft 6 is pushed so as to make the cylindrical distal body 5 come out of the external catheter 2.

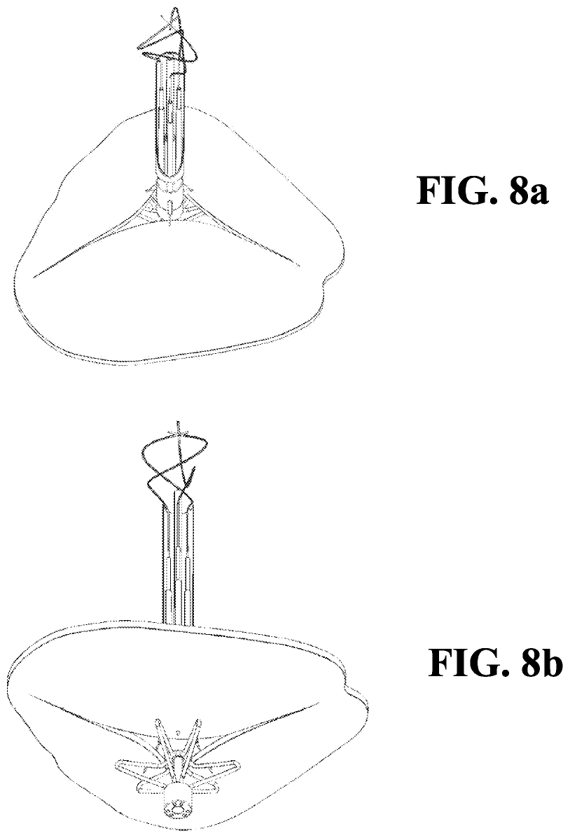

Since the arms 7 outside the external catheter 2 are free to expand, they assume a radially expanded position with an elbow bend 8, define radially oriented coplanar supports to sustain the flaps of a tricuspid or mitral valve. As shown in FIGS. 8a and 8b, the flaps of the tricuspid valve rest on the bent arms 7. By pulling the rigid coaxial shaft 6, a surgeon may raise the bent arms 7 so as to bring the flaps back into the valve plane. The device 1 of this disclosure is therefore structured to support the valve flaps without interfering with the chordae tendineae of the valve itself (not shown in the figures) which are located on the opposite side with respect to the bent arms 7.

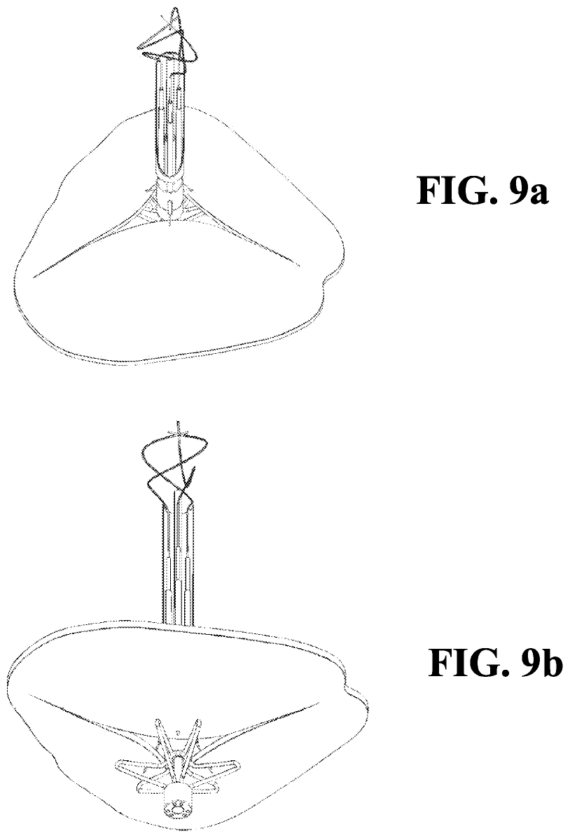

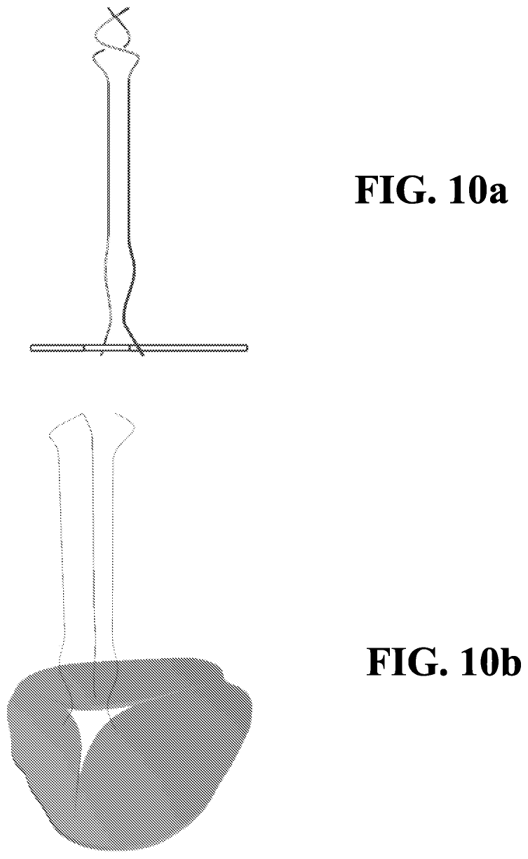

When the flaps are aligned with the plane of the valve, suture threads 11, inserted into the internal catheters 10, are made to come out of the holes in the lateral surface of the cylindrical proximal body 3 so as to capture the flaps, as shown in FIGS. 9a and 9b. Once the flaps have been captured, the device 1 is removed leaving the suture threads 11 as shown in FIGS. 10a and 10b. Subsequently, by means of known tools or the cutting and holding device 12, the suture threads are tied together and cut at a desired length, so that the valve flaps remain tied together.

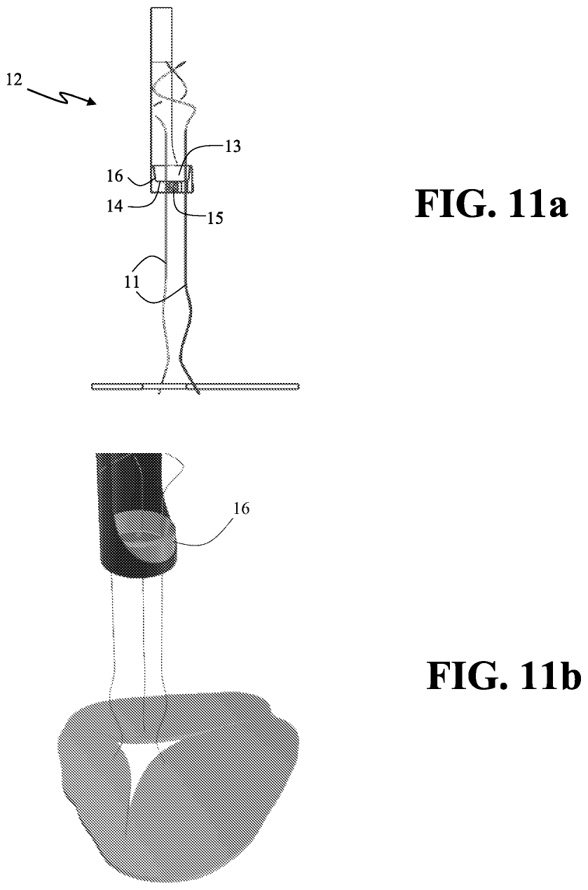

According to one aspect, the suture threads can be stretched and cut at a desired length by means of a cutting and holding device 12 of suture threads of the type illustrated in FIGS. 11a to 14c. According to one aspect, it comprises at least one internally threaded countersunk washer 13, having a flat bottom plate 14 with a central axial through hole 15 with nut thread, which crosses it perpendicularly, and having a side wall 16 which rises from the flat bottom plate 14 along a peripheral area of the flat plate 14 itself. In the flat bottom plate 14 there is at least a second through hole 17, in an off-center position with respect to the axial central through hole 15, configured to be crossed by at least one suture thread 11. The countersunk washer 13 is configured to be inserted into the external catheter, which optionally can be the same external catheter 2 of the device 1, keeping the central through hole 15 coaxial to the catheter itself. As shown in FIGS. 11a and 11b, from the outside of the patient's body the countersunk washer 13 is inserted into the catheter with at least one suture thread 11 running in the respective second through hole 17.

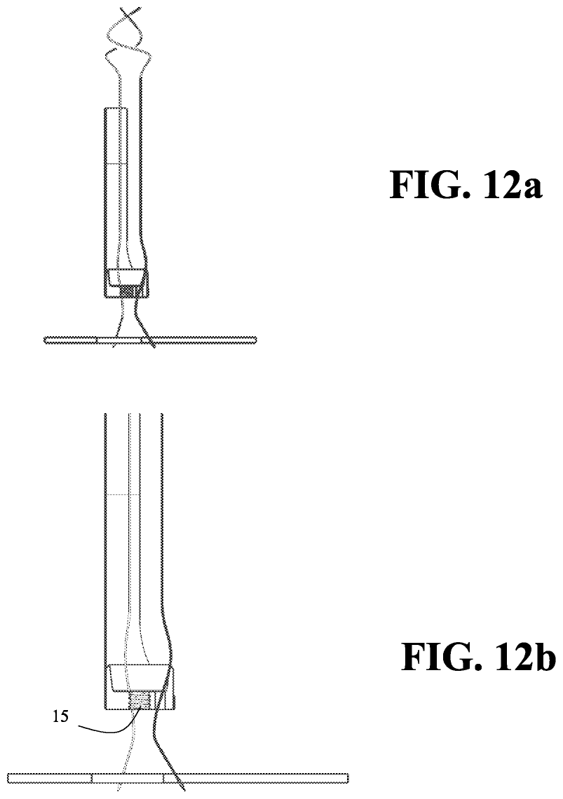

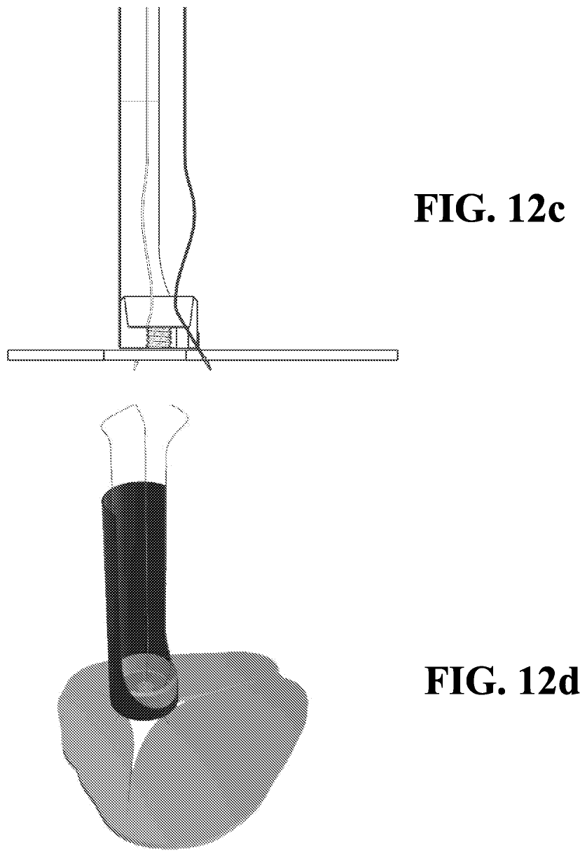

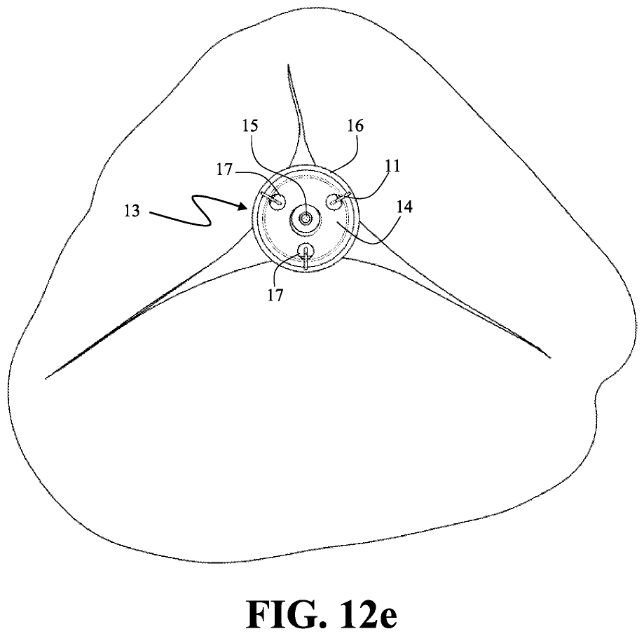

From the outside of the patient's body (FIGS. 11a to 12e) the suture threads 11 are inserted into the holes 17 of the countersunk washer 13 and the countersunk washer 13 is guided to the center of the valve to be repaired (FIG. 12e).

Preferably, on the flat plate 14 at the bottom of the countersunk washer 13 there are as many second through holes 17 as the suture threads 11 to be stretched and cut, so that each suture thread is inserted into the respective second through hole 17. In the example shown in FIG. 12e, the flat bottom plate 14 of the countersunk washer 13 has holes for the suture threads 11 arranged with angular symmetry with respect to the central through hole 15 of the countersunk washer 13. An advantage of the symmetrical arrangement consists in the fact that, by stretching the threads at the same time, the countersunk washer 13 automatically positions itself so that its central through hole 15 is in the middle of the threads 11. However, it is possible to make only one hole 17 (distinct from the central through hole 15) to pass all the threads 11 through it, but in this case the position of the central through hole 15 will not be centered between the suture threads 11.

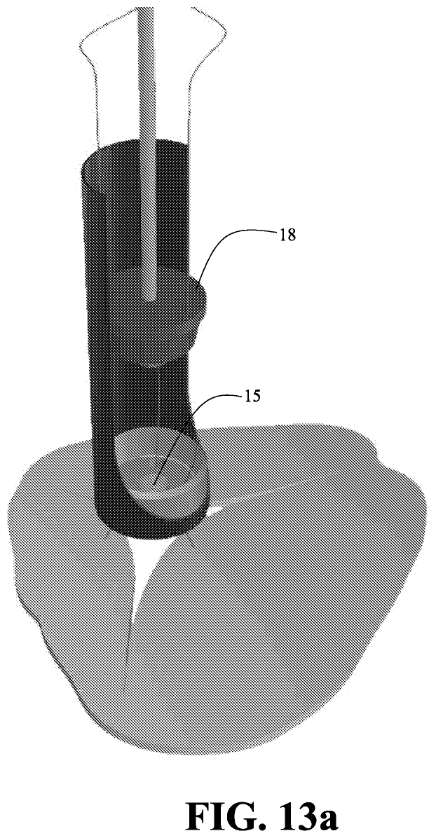

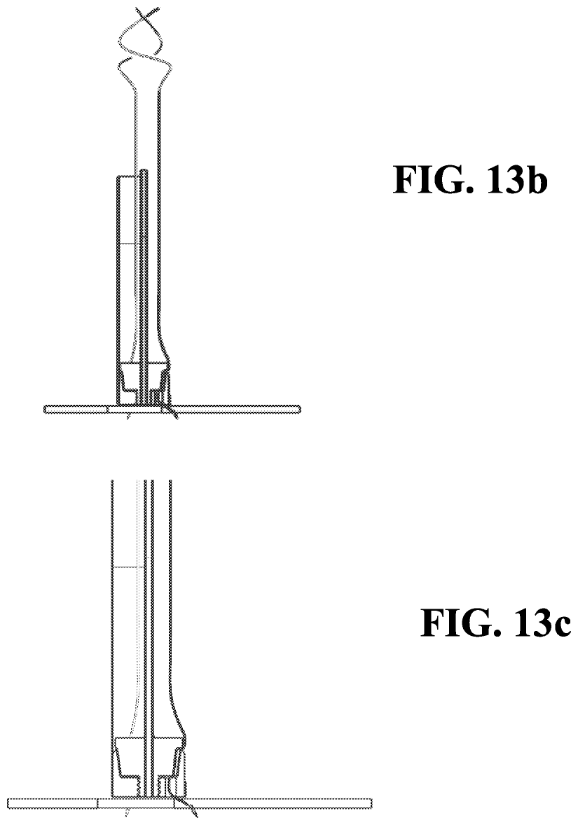

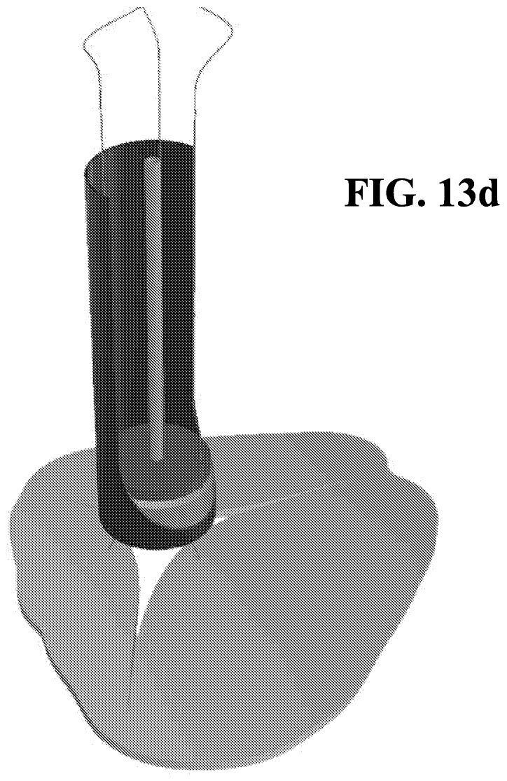

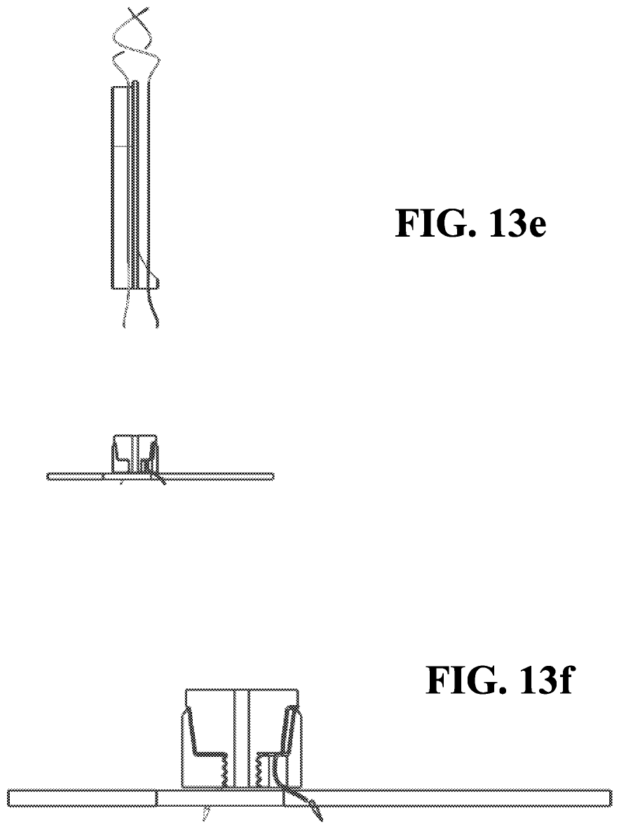

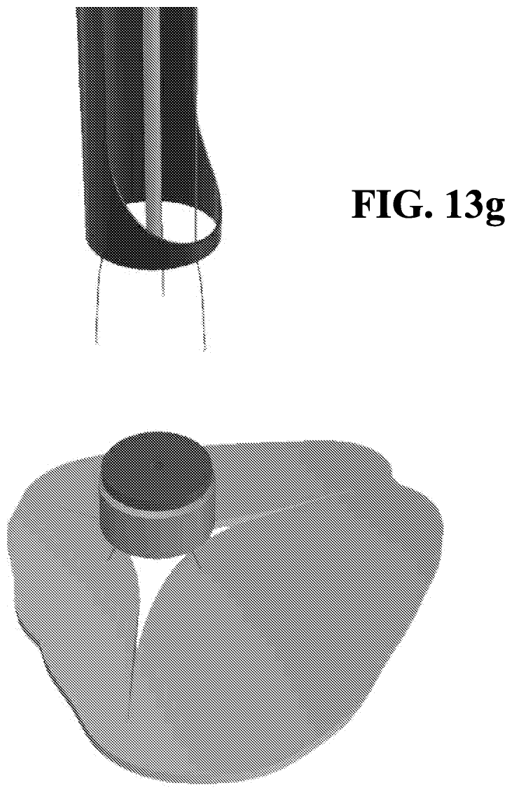

Once the countersunk washer 13 is substantially positioned in the valve plane, the threads are pulled axially with respect to the catheter so that the flaps are extended in the valve plane and a screw is coaxially advanced into the catheter (FIGS. 13a to 13d) having a stem 19 which can be screwed into the central through hole 15 and having a head 18 configured to rest on a free edge of the side wall 16 of the countersunk washer 13. By tightening the screw in the internally threaded countersunk washer 13, the head of the screw 18 is pressed against the free edge of the countersunk washer 13 and together cooperate to cut the suture threads 11, which remain trapped between them (FIGS. 13e to 13g) keeping the flaps extended.

Optionally, the free edge of the side wall 16 of the countersunk washer 13 will have a profile shaped like a blade so as to facilitate the cutting of the suture threads 11 when the screw is tightened, as can be seen in the detail views of the figures from 14a to 14c.

Once the suture threads 11 have been cut, the external catheter and the suture threads are withdrawn, leaving the countersunk washer 13 and the screw tightly screwed therein in the valve plane.

In the embodiment illustrated in the figures, the device 1 has three pairs of arms 7 so that each flap of the tricuspid valve is supported by at least one pair of arms 7.

However, there may also be more than six arms 7, should a greater support of the tricuspid valve flaps be required, or there may be only two pairs of arms 7, for example to support the two flaps of a mitral valve. Similarly, there can also be only two internal through channels 9 in the cylindrical proximal body 3, to catch the two flaps of a mitral valve with as many suture threads that protrude from respective two holes in the lateral surface of the cylindrical proximal body 3.

According to one aspect, the 7 arms are made of Nitinol. Optionally, they are composed of elastic material which gives an elbow-bent configuration at rest, as illustrated in FIGS. 5a and 5b, and can be configured in such a way as to be elastically forced to remain extended when they are inside the external catheter 2.

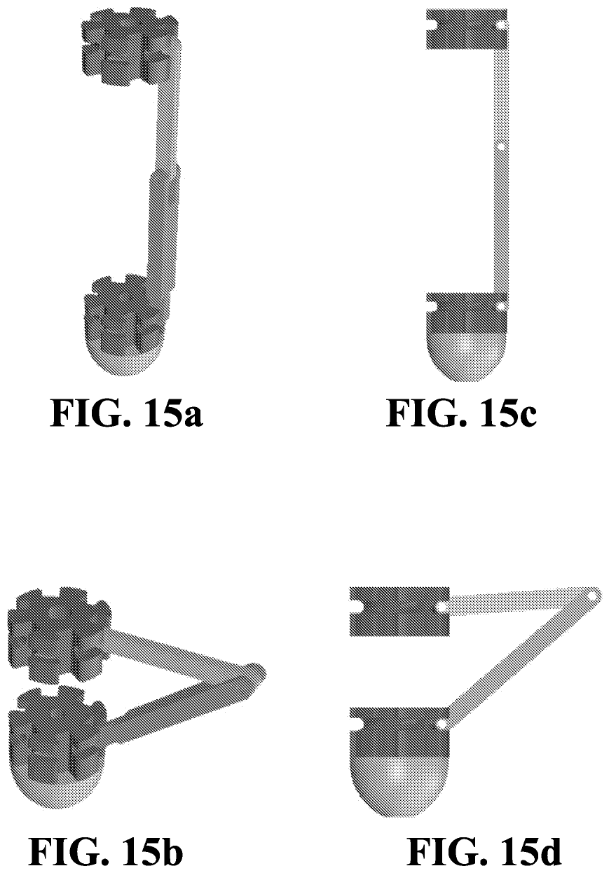

According to one aspect shown in FIGS. 15a to 15d, each arm is made by hinging a first rigid shaft at the first end of the arm on the second base surface of the cylindrical proximal body, and a second rigid shaft at the second end of the arm to the cylindrical distal body. The two shafts are hinged to each other at an intermediate area so that each arm can be bent forming the elbow 8 at the point where the two rods are hinged to each other. As in the case illustrated in FIGS. 9a and 9b, the bent arms define coplanar supports of the flaps of a heart valve.

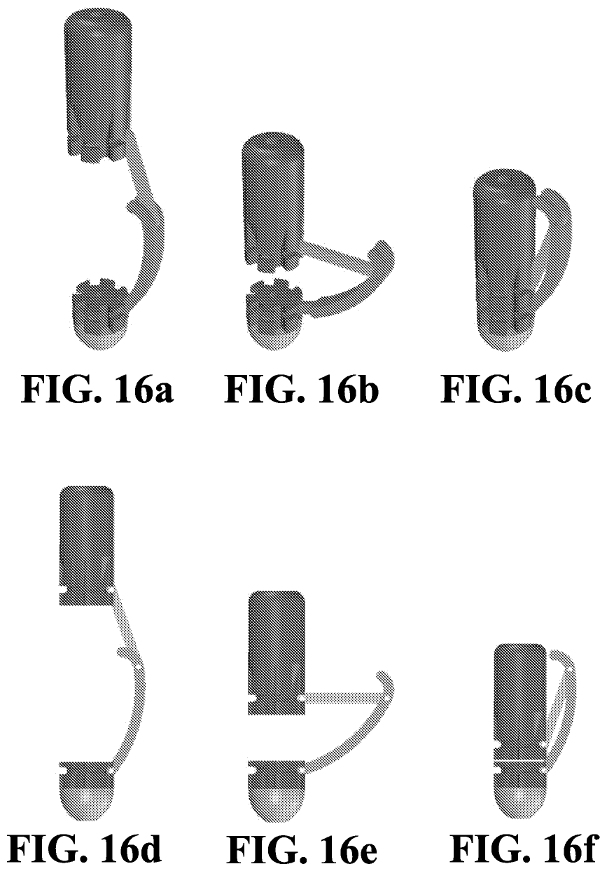

According to one aspect shown in FIGS. 16a to 16f, the arms are made with two rigid rods hinged to each other as in FIGS. 15a to 15d but, unlike the latter, the shaft connected to the cylindrical distal body 5 has a free end shaped as a hook. As indicated in the succession of FIGS. 16a to 16f, by progressively approaching the cylindrical proximal body 3 to the cylindrical distal body 5, the arm passes from the extended position (FIGS. 16a and 16d) to the radially expanded position (FIGS. 16b and 16e) forming an elbow 8, and further to a closed position (FIGS. 16c and 16f) in which the hook-shaped free end is functionally configured to cooperate with the coaxial shaft 6 to tighten a flap of a heart valve like two jaws. By making the arms as illustrated in FIGS. 16a to 16f, it is possible to keep the flaps flat (FIGS. 16b and 16e) or to pull them with the hook-shaped ends towards the coaxial shaft 6 by moving the proximal body 3 towards the distal body 5 up to tighten the flaps between the coaxial shaft 6 and the hook-shaped end.

An advantage in making the arms in this way is the fact that, when the flaps are tightened between the coaxial shaft 6 and the respective hook-shaped end, they are well stretched and cannot move, making it easier to join them.

A further advantage is the fact that the flaps are pulled more towards the center of the valve plane and can be crossed by the respective suture wires in an area further away from the free end of the flap. Otherwise, the area in which the flaps are crossed by the respective suture threads will be determined by the inclination with which the suture threads exit from the respective outlet holes of the through channels 9 in the lateral surface of the cylindrical proximal body 3.

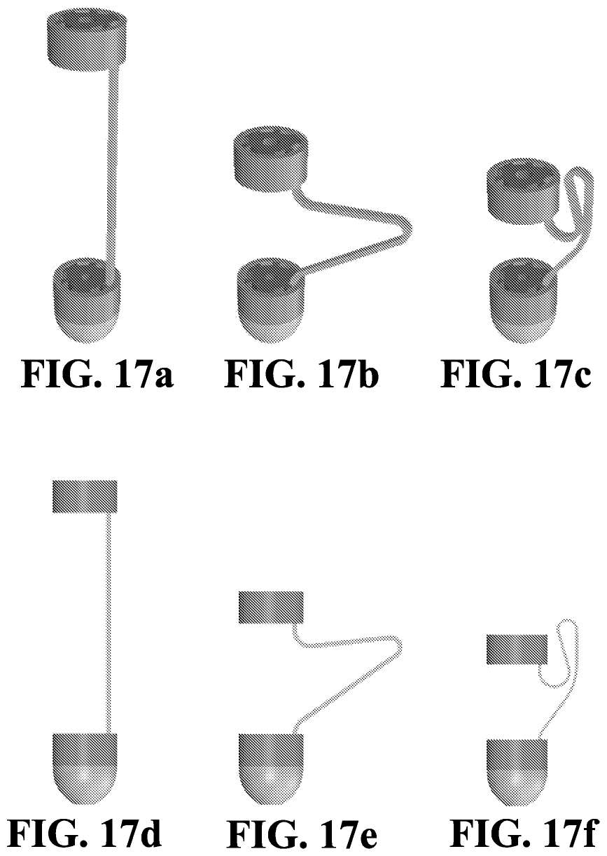

According to an aspect shown in FIGS. 17a to 17f, the arms are not constituted by two rigid hinged rods but are made in one piece with a shape memory material or with an elastically deformable material. As for the embodiment shown in FIGS. 16a to 16f, each arm is configured so that, progressively approaching the cylindrical proximal body 3 to the cylindrical distal body 5, the arm passes from the extended position (FIGS. 17a and 17d) to the radially expanded position (FIGS. 17b and 17e) forming the elbow 8, and further to the closed position (FIGS. 17c and 17f) in which the elbow 8 is functionally configured to cooperate with the coaxial shaft 6 to tighten a flap like two jaws of a heart valve.

The device 1 for joining the flaps of a tricuspid or mitral valve can optionally be distributed as part of a kit for surgical operations, comprising the device 1 itself and a device 12 for cutting and holding at least one suture thread, comprising:
an external catheter 2 adapted to be inserted into a patient's blood vessel;
an internally threaded countersunk washer 13, having a flat bottom plate 14 with a central axial through hole 15 with a nut thread which crosses substantially perpendicularly the flat plate 14, and having a side wall 16 which rises from the flat bottom plate 14 along a peripheral region of the flat bottom plate 14, the flat bottom plate 14 having at least a second through hole 17 configured to be crossed by at least one suture thread 11, the countersunk washer 13 being configured to be inserted into the external catheter 2 while maintaining the central through hole 15 coaxially with the external catheter 2;
a screw configured to coaxially advance into the external catheter, having a shaft 19 screwable into the central through hole 15 and having a head 18 configured to rest on a free edge of the side wall 16 of the countersunk washer 13 and to cooperate with the free edge of the countersunk washer 13 to cut the suture thread held between the countersunk washer 13 and the head 18 of the screw.

According to one aspect, the cutting and holding device 12 of at least one suture thread 11 could be distributed separate from the catching device 1 of this disclosure and used in other devices to stretch and cut suture threads during surgical operations.

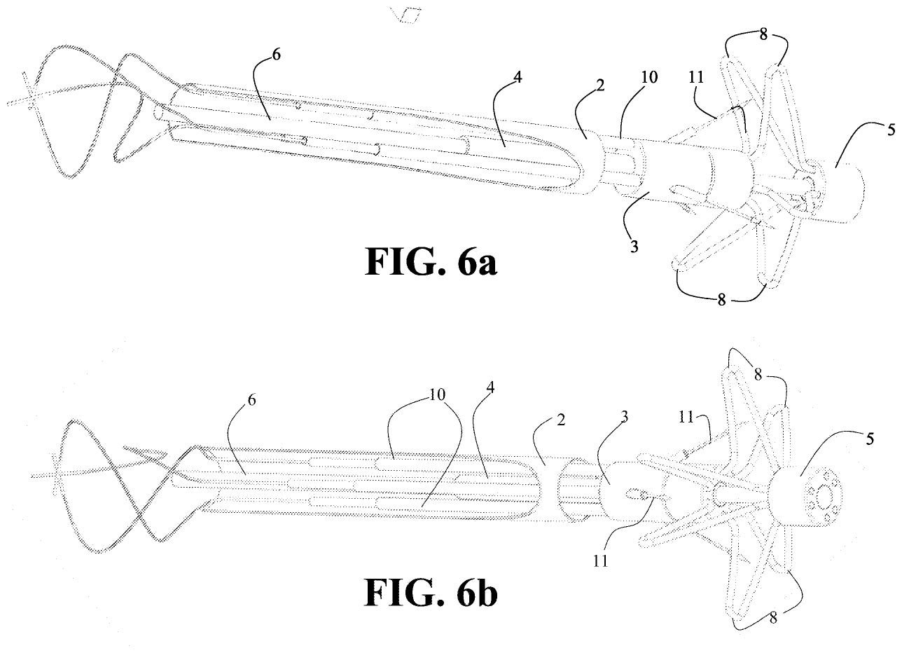

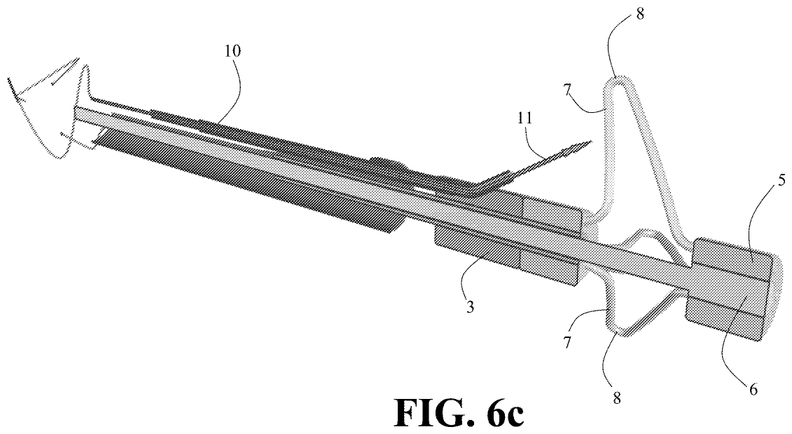

FIGS. 6a to 6c show suture threads ending with harpoons shaped like an arrow so as to cross the flaps of a heart valve from one side while remaining hooked on the opposite side, thus allowing the flaps to be pulled in a radial direction by pulling the threads.

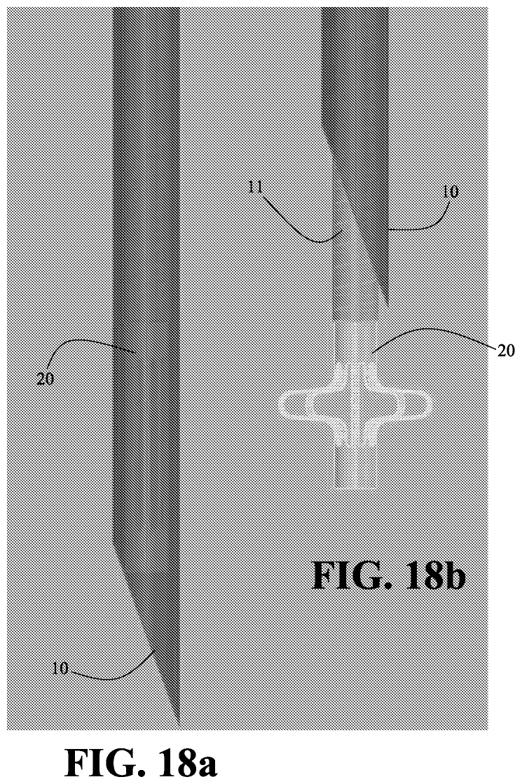

FIGS. 18a and 18b show an alternative embodiment in which the harpoon of each suture thread is replaced with a hooking element 20, visible in FIG. 18a in a longitudinally extended configuration in semitransparency inside a carrying catheter 10 and in FIG. 18b on the outside of a carrying catheter 10 in a radially expanded configuration. Unlike the harpoons shown in FIGS. 6a to 6c, in the embodiment of FIGS. 18a and 18b the carrying catheter 10 is shaped like the tip of a syringe needle so as to pierce a flap of a heart valve by entering from one face and release the hooking element 20 on the opposite face of the flap. The hooking element 20 is configured so as to remain longitudinally extended (FIG. 18a) when it is inside the carrying catheter 10, and to expand (FIG. 18b) radially when it is free. Once the hooking element 20 is free to expand, it can no longer pass through the hole made in the heart valve flap so that it remains hooked to the flap and allows it to be pulled.

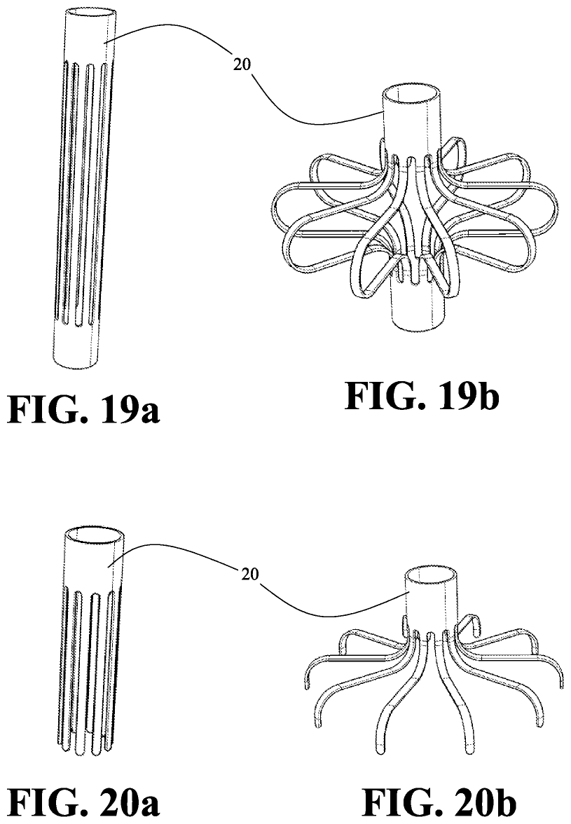

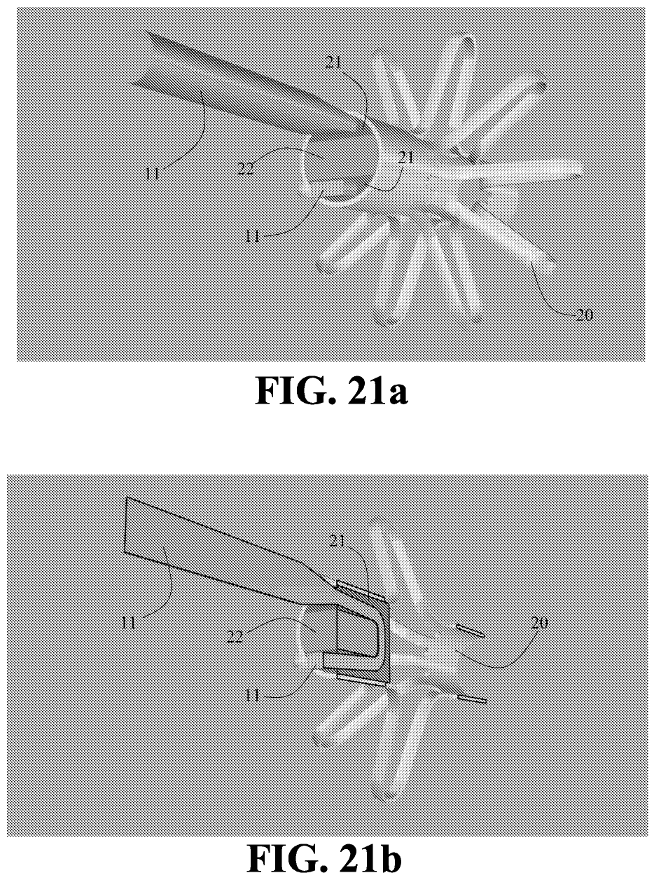

FIGS. 19a and 20a show possible embodiments of such a hooking element 20, both obtained by cutting a tubular body made of elastic or shape memory material into longitudinal strips. In FIGS. 19a and 20a the longitudinal strips are in the longitudinally extended configuration when they are in the supporting catheter, and are configured to automatically expand radially when they are outside the internal supporting catheter 10. FIGS. 19b and 20b show the corresponding hooking element 20 of FIGS. 19a and 20a in a radially expanded configuration. The shape that the hooking elements 20 can take in the expanded configuration can also be different from the one shown in the figures. For example, the hooking element of FIG. 20b can assume a radially expanded configuration in which the strips form curls or arrange themselves to remain radially extended.

According to one aspect, each hooking element 20 of FIGS. 20a and 20b can be fixed to a respective suture thread 11 in the manner illustrated in FIGS. 21a and 21b. As shown in the sectional view of FIG. 21b, the suture thread 11 is embedded in a solid resin block 21. In turn, the solid resin block 21 is interlocked to the internal walls of the anchoring element by means of a central body 22 wedged in the solid resin block.

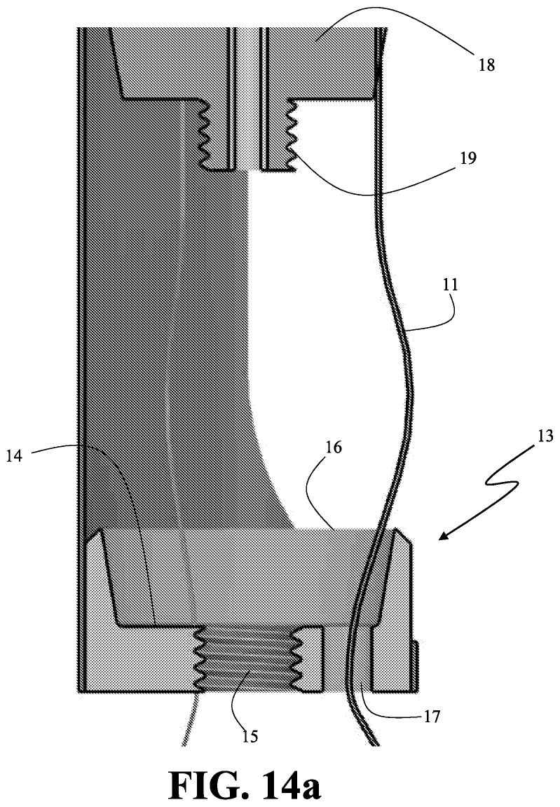

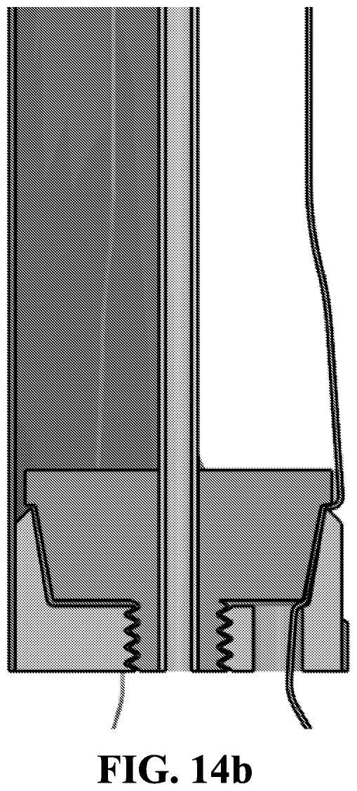

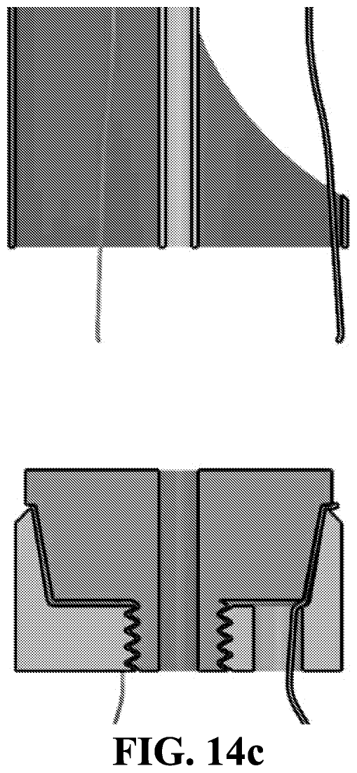

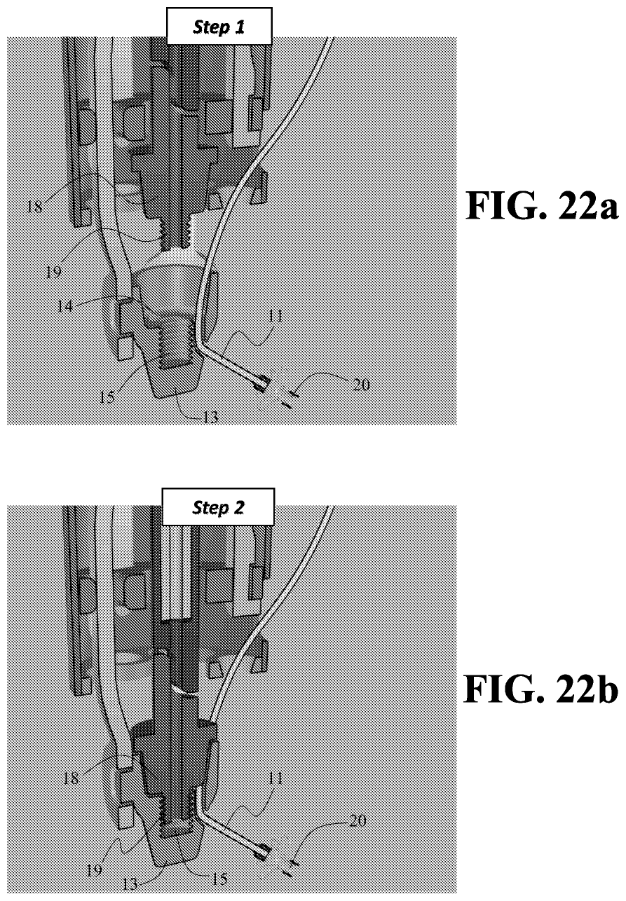

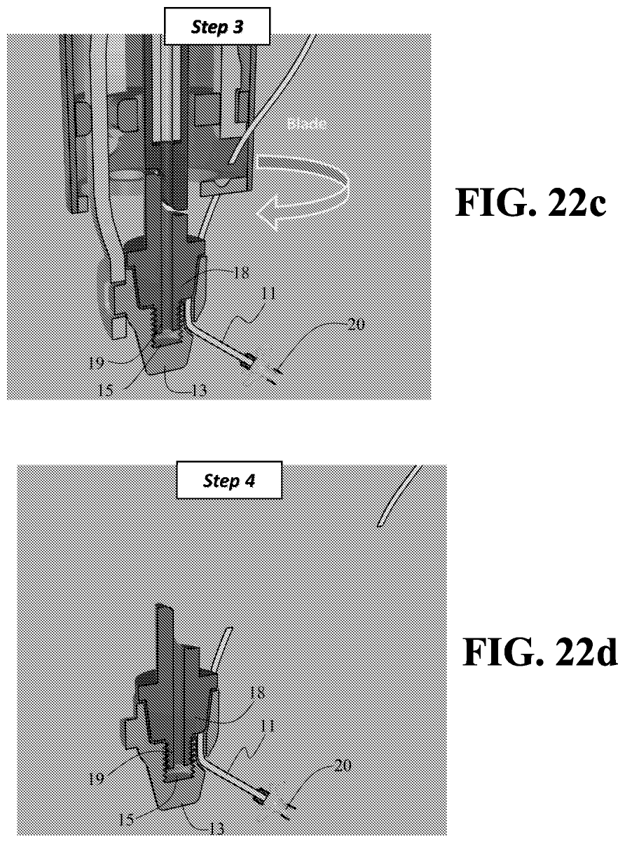

According to one aspect, a cutting and holding device according to another form of the present disclosure can be made in the manner illustrated in FIGS. 22a to 22d. The operation of this other embodiment is similar to that of the cutting and holding device shown in FIGS. 10a to 14c and can be used with the device of FIG. 6a. The device of FIGS. 22a to 22d is also based on the cooperation between a screw and a countersunk washer 13 with a central hole 15, which in the figures is a blind hole 15, with a nut screw thread. Unlike the device of FIGS. 14a to 14c, in the embodiment illustrated in FIGS. 22a to 22d the countersunk washer 13 with a central hole 15 and the screw inserted therein serve only to hold the suture threads and not to cut them. The suture threads are cut because the illustrated cutting and holding device has a blade (FIG. 22c) which can rotate circumferentially to sever the threads protruding from the countersunk washer (FIG. 22d).

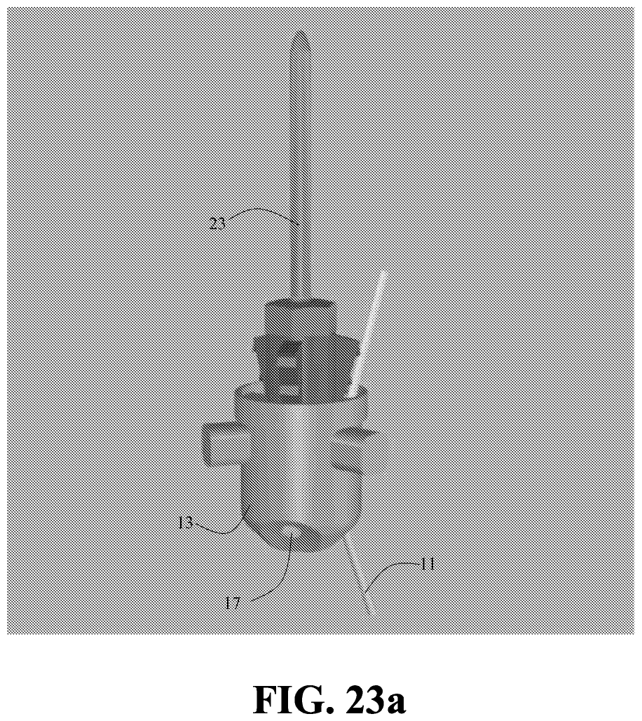

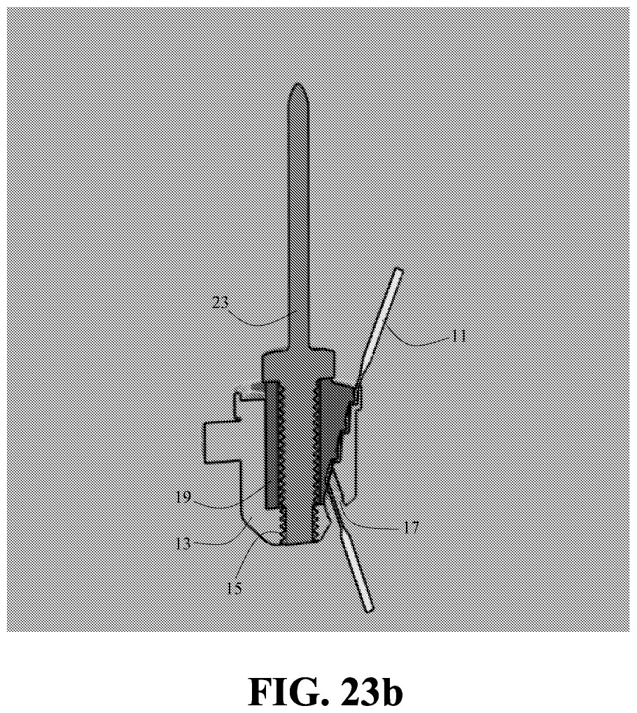

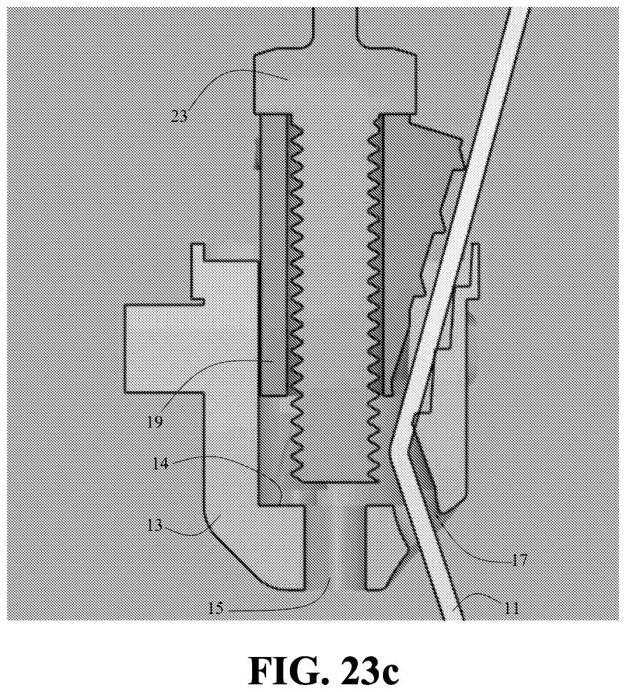

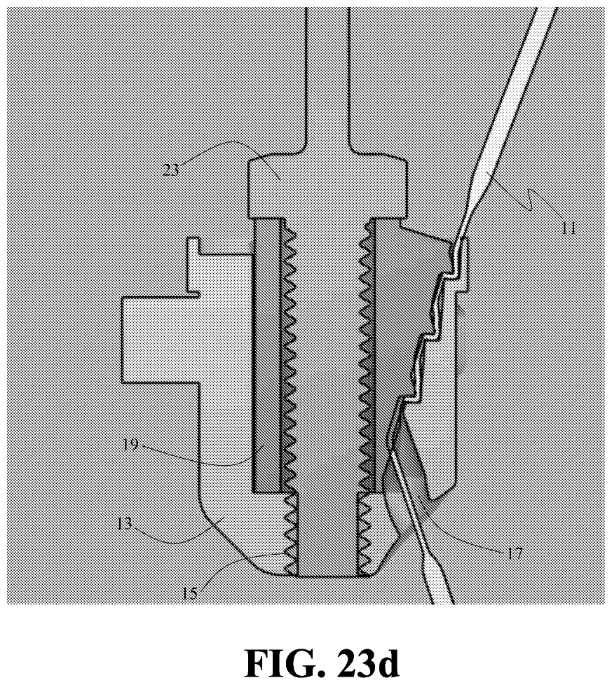

According to yet another embodiment of the cutting and holding device illustrated in FIGS. 23a to 23d, the stem 19 of the screw inserted in the central through hole 15 of the washer 13 is hollow and internally threaded with a nut screw. Thanks to a threaded pin 23, the screw can be simply inserted into the hole of the washer and firmly joined to the washer 13 by screwing the threaded pin 23 into the nut screw hole of the washer 13.

Once this operation is finished, the suture threads are cut with a blade which can be rotated circumferentially as in the device of FIG. 22c and the threaded pin 23 is left in place (FIG. 23b).

According to one aspect shown in the sectional views of FIGS. 23c and 23d, the countersunk washer 13 has an internal stepped profile mating with an external stepped profile of the screw, so as to crush and hold the sutures in several points, preventing them accidentally slipping off.

Any variations or additions can be made by experts in the technical field to the embodiments described and illustrated here, while remaining within the scope of the following claims. In particular, further embodiments may include the technical characteristics of one of the following claims with the addition of one or more technical characteristics described in the text or illustrated in the figures, taken individually or in any reciprocal combination.

1. A device for joining heart valve flaps, comprising:
an external catheter adapted to be inserted into a patient's blood vessel;
a cylindrical proximal body, defining a central axial cavity which runs in the cylindrical proximal body from a first base surface to a second base surface;
a cylindrical distal body, having a rigid coaxial shaft which is fixed and rises from a central position of a base surface of the cylindrical distal body, in which said coaxial shaft is threaded slidingly in said central axial cavity of the cylindrical proximal body;
a plurality of arms, each arm of the plurality of arms having a first end attached to the second base surface of the cylindrical proximal body and a second end attached to said cylindrical distal body, wherein each arm is configured to assume a radially position expanded with an elbow bend when outside said external catheter, and to assume an extended position parallel to said rigid coaxial shaft when both the cylindrical proximal body and the cylindrical distal body are inside said external catheter, in which said arms are configured so that, when they are in said radially expanded position with an elbow bend, they define radially oriented supports to support flaps of a heart valve;
wherein said cylindrical proximal body defines a plurality of through channels which run in the cylindrical proximal body from as many inlet holes in the first base surface of the cylindrical proximal body, to as many outlet holes in a surface lateral of the cylindrical proximal body;
a plurality of internal catheters, each internal catheter of said internal catheters is connected to a respective inlet hole of said inlet holes and is configured to convey a respective suture thread to a respective through channel of said channels loops and to let said suture thread partially in said radial direction from the respective outlet hole from the lateral surface of the cylindrical proximal body;
a plurality of suture threads, in which each suture thread of said suture threads is inserted into a respective internal catheter of said internal catheters, said suture threads having terminations with hooking elements configured in such a way as to hook the flaps of said heart valve when made to come out of the holes of the cylindrical proximal body and in such a way as to expand autonomously in the radial direction after having harpooned said flaps so that, by pulling the suture threads, the valve flaps are dragged by said hooking elements, approaching one another towards a central longitudinal axis of the device.

2. The device according to claim 1, wherein each arm of the plurality of arms defines coplanar supports.

3. The device according to claim 1, wherein each arm of the plurality of arms is integral and consists either of:
a shape memory material; or
an elastically deformable material.

4. The device according to claim 3, wherein each arm of the plurality of arms is configured so that, by progressively approaching the cylindrical proximal body to the cylindrical distal body, the arm passes from said extended position to said radially expanded position with said elbow bend and further to a closed position in which said elbow bend is functionally configured to cooperate with said coaxial shaft to tighten as two jaws a flap of a heart valve.

5. The device according to claim 1, wherein each arm of the plurality of arms (7) consists of a first rigid shaft hinged at said first end of the arm on the second base surface of the cylindrical proximal body, and a second rigid shaft hinged at said second end of the arm to said cylindrical distal body, said first shaft and said second shaft being rigid and hinged to each other at an intermediate zone between said first end and said second end.

6. The device according to claim 5, wherein said second shaft has a free end in the shape of a hook, each arm being configured so that, progressively approaching the cylindrical proximal body to the cylindrical distal body, the arm passes from said extended position to said radially expanded position with said elbow bend and further to a closed position in which said hook-shaped free end is functionally configured to cooperate with said coaxial shaft to tighten like two jaws a flap of a heart valve.

7. The device according to claim 1, wherein each hooking elements of said hooking elements is composed of a tubular body partially cut into longitudinal strips, in which said longitudinal strips are made of elastic or shape memory material and are configured to remain extended longitudinally when they are in the respective internal catheter and to expand autonomously in the radial direction when they are outside the respective internal catheter.

8. The device according to claim 7, wherein said internal catheters have terminations shaped like a tip of a syringe needle and are configured to pierce a flap of a heart valve.

9. The device according to claim 1, wherein said plurality of through channels comprises three through channels and said plurality of internal catheters comprises three internal catheters each of which is connected to an inlet hole of the respective through channel.

10. The device according to claim 1, comprising cutting means configured for cutting said suture threads.

11. A kit for surgical operations, comprising a device for joining flaps of a heart valve according to claim 1 and a device for cutting and holding a suture thread, wherein said device for cutting and holding includes:
an internally threaded countersunk washer, having a bottom flat plate with a central axial through hole with a thread that crosses perpendicularly said flat plate, and having a side wall which rises from said flat bottom plate along a peripheral area of the flat bottom plate, said flat bottom plate having a second through hole configured to be crossed by a suture thread, said countersunk washer being configured to be inserted in said external catheter while maintaining said central through hole coaxially to the external catheter;
a screw configured to coaxially advance in said external catheter and to be constrained to the countersunk washer so as to retain said suture thread sandwiched between an internal surface of the side wall of the countersunk washer and an external surface of said screw.

12. The kit according to claim 11, wherein said screw has a stem screwable into said central through hole and having a head configured to rest on a free edge of the side wall of the countersunk washer and to cooperate with said free edge of the countersunk washer to cut said suture thread sandwiched between the countersunk washer and the head of the screw.

13. The kit according to claim 11, wherein said screw has a stem which can be screwed into said central through hole and said cutting and holding device comprises a blade configured to be rotated circumferentially in a plane orthogonal to an axial direction of said countersunk washer to sever the suture threads which protrude from the countersunk washer.

14. The kit according to claim 11, wherein:
said screw has an axial through hole internally threaded with a nut screw, corresponding to the central axial through hole of the internally threaded countersunk washer;
said cutting and holding device comprises:
a threaded pin configured to be screwed simultaneously into the axial through hole of said screw and into the central axial through hole of the countersunk washer to fix said screw to said countersunk washer,
a blade configured to be rotated circumferentially in a plane orthogonal to an axial direction of said countersunk washer to sever the suture threads protruding from the countersunk washer.

15. The kit according to claim 11, wherein the countersunk washer has an internal stepped profile mating with an external stepped side profile of said screw, so as to crush and hold in several points a suture thread of said suture threads preventing the suture thread from accidentally unthreading.
